(19) United States
(12) Patent Application Publication
(10) Pub. No.: US 2007/0099278 A1
(43) Pub. Date: May 3, 2007

(54) Production of biodiesel from combination of corn (maize) and other feed stocks

(76) Inventor: Palaniswamy Ramaswamy Aare, Hyderabad (IN)

Correspondence Address: PALANISWAMY RAMASWAMY AARE;HIG 413,, BHEL TOWNSHIP, R.C. PURAM, HYDERABAD, AP, 502032, IN

(21) Appl. No.: 11/590,262

(22) Filed: Oct. 31, 2006

Related U.S. Application Data
(60) Provisional application No. 60/732,264, filed on Nov. 1, 2005.

(51) Int. Cl. C12P 7/64; C12P 7/06; C07C 51/43

(52) U.S. Cl. 435/134; 435/161; 554/174

(57) ABSTRACT

A method and system to produce biodiesel from a combination of corn (maize) and other agro feedstock may be simarouba, mahua, rice, pongamia etc. Germ is separated (either by wet process or dry process) from corn, crude corn oil extracted from germ and corn starch milk/slurry is heated and cooked in jet cooker to about 105 degree Celsius, enzymes added to convert starch into fermentable sugars in liquification and saccharification process and rapidly cooled down to about 30 degree Celsius. Simarouba fruits syrup, mahua syrup is mixed with corn starch milk (after saccharification). When yeast is added the fermentation takes place for about 72 hours. Thereafter the fermented wash is distilled to produce ethanol. Water consumed in dry process is very less compared to traditional wet process system. Corn oil and mixture of other oils is fed into transesterification (reaction) vessels where ethanol with catalyst, usually sodium hydroxide is added and reaction takes place for about a period of 2-8 hours. Crude biodiesel and crude glycerin as by-products is produced. Excess ethanol removed by distillation process. Crude biodiesel washed with warm water to remove residual soaps or unused catalyst, dried and biodiesel stored for commercial use. Oil extracted from spent bleach mud (used sodium bentonite), a waste product of edible oil refineries may also be utilized for economical production of biodiesel in combination of corn oil and ethanol.

### CROSS-REFERENCE TO RELATED APPLICATIONS

This application claims the benefit of priority of U.S. Provisional Patent Application Ser. No. 60/732,264, filed Nov. 1, 2005 and entitled “PRODUCTION OF BIODIESEL FROM CORN (MAIZE) AND OTHER FEEDSTOCK”, the subject matter of which is hereby incorporated by reference herein.

This invention relates generally to biodiesel production and more specifically, to a novel method that uses corn (maize), other selected crops (cereals, paddy), oil bearing seeds and relevant industrial by-products for the production of Biodiesel (Ethyl esters) and clean burning, low emission alternative fuel.

### BACKGROUND OF INVENTION

Biodiesell (Ethyl Esters) Production

Ethyl esters, also called Biodiesel is an oxygenerated fuel has numerous industrial and commercial applications. For instance, it is used to blend with petroleum diesel for use in diesel engines and it is also used as boiler fuel.

Ethyl esters are derived from three primary processes:

Extraction of corn oil

Fermentation of sugars (corn starch) to convert into ethyl alcohol

Transesterification of oil, ethanol (ethyl alcohol) with catalyst.

Extraction of oil involves the breaking down of complex organic substances of corn (Zea Mays Indentata, Zea Mays Indurata) into germ, starch, protein and fiber. Germ which contains 80-85% of corn oil, with combination of mechanical and solvent processes, oil is extracted from the germ.

Starch component of corn is subjected to enzyomatic processes and yeast is added for fermentation. Fermentation involves the breaking down of the complex organic substances into simpler ones. Alcohol fermentation is enabled by several types of bacteria and yeast through a simple enzymatic anaerobic action, yeast converts sugar molecules into alcohol and carbon dioxide.

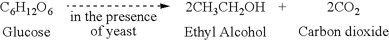

The above fermentation reaction is the basic method in the production of alcohol through fermentation. Glucose (a type of sugar) is broken down into ethyl alcohol and carbon dioxide by yeasts. The above reaction is a simplified one that actually involves intermediate steps its conversion into acetaldehyde and finally to ethyl alcohol (Ethanol). The above fermentation reaction generally yields less than 10% of ethanol. To obtain higher concentrations of alcohols, this ethanol is subjected to a distillation process.

Most bio oils are triglycerides (TGS; Triglyceride: TG) chemically TGS are Triglyceryl esters of various fatty acids with glycerol.

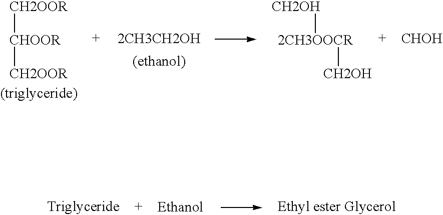

The derivatives of TGs for fuels are ethyl esters. These are formed by Transesterification of the TG with ethanol in the presence of a basic catalyst usually Sodium Hydroxide (NaOH) or Potassium Hydroxide (KOH). The conversion of component TG to simple alkyl ester (transesterification) with ethanol (ethyl alcohol) reduces the high viscosity of oils & fats.

OILSEEDS, CORN & RICE (PADDY) FOR PRODUCTION OF BIODIESEL: Soyabean, Canola, Coconut, Cottonseed, Linseed, Palm, Peanut, Rapeseed, Sunflower, Safflower, Simarouba [simarouba glauca DC], Mustard, Paddy (rice bran oil) and Corn are raw materials for the edible oil production. Jatropa [jatropa curcas; wild caster seeds], pongamia [pongamia pinnata (L) pierre, pongamia glabra vent, pongam karanga are one of the nitrogen fixing trees (NFTS)] to produce seeds containing 30 to 40 percent oil], Mahua [maduca indica; maduca longifolia seeds], Neem [margosa] are raw materials for inedible oil production.

Simarouba, Soyabean, Canola, Coconut, Palm, Cottonseed, Linseed, Peanut, Rapeseed, Mustard hybrids, Jatropha (inedible) Pongamia (inedible) contains high oil content compared to Corn and Rice (paddy) (12%-23%), but very less starch. Hence extraction of oil is economical, but the major disadvantage is the extraction of alcohol from the oilseeds (edible & inedible), which results in depending on the ethanol or methanol for production of Biodiesel. Oilseeds are having limitations in the production of Biodiesel due to inherent weakness of non or very less availability (uneconomical) starch. Corn (maize) and paddy (rice) contains less percentage of (extractable) oil. However it contains high starch & carbohydrates, hence production of Ethanol and Biodiesel is made easy as one source of raw material. Mahua seeds (Maduca indica; Maduca longifolia seeds) contains appreciable oil content (33%-38%) and its flowers (dried) contains 70%-72% sugars, hence production of Biodiesel is very economical as one source raw material. Simarouba (simarouba glauca DC) contains appreciable high oil content (60%-70%) and its fruits contain 11% sugars, hence production of Biodiesel is very economical as one source of raw material.

CORN: Corn (Zea Mays Indentata, Zea Mays Indurata) is grown in different parts of the world, generally as a pure crop for commercial grain production. Corn is resourceful in that it contains high starch percentages and low oil percentages (3%-4%). Hence it is very useful for extraction of oil and ethanol. Primary raw materials for Biodiesel production are oil (TG) and ethanol (ethyl alcohol)/methanol. Corn protein and fiber are useful in animal feed production.

Biodiesel produced from corn contains less sulphur content and hence produce more pure cleaner Biodiesel. Production of Biodiesel, generally made at the rate of 6:1 to 7:1, ethanol and oil which ideally suits for corn.

360 to 400 litres of ethanol can be produced from one ton of corn grains.

SIMAROUBA: Simarouba (Simarouba glauca DC), known as paradise tree or energy tree, aceituno, commonly known as simarouba is an oil bearing tree. This is tropical tree grows from sea level to upto 1000 mts. above sea level in all types of soil (with well drainage facilities) with pH 5.5 to 9.0, establishes well in 400 mm to 4000 mm annual rainfall. Fruits of Simarouba contains 11% fermentable sugars and seeds contains 60% to 70% oil (edible). 160 plants can be planted per acre and Simarouba produces about 3200 Kgs of fruits and about 1100 Kgs of oil seeds per annum/per acre (8-9 years old trees). Thus, the oil yield will be about 800 Kgs./acre and 110-140 litres of Ethanol per ton of fruit (on fermentation and distillation process). Simarouba tree yield fruits until the age of 55-60 years.

MAHUA: Mahua (Maduca indica; Maduca longifolia) are two species which are abundantly available in India, commonly known as Madhuva. The two varieties of Mahua (Maduca indica; Maduca longifolia) is closely related and no distinction can be made of their seed or oil. The seeds when subjected to expeller for oil extraction, will yield 33%-38% of oil. Dried flowers of Mahua (Maduca indica; Maduca longifolia) contains 70%-72% fermentable sugars which yield nearly 400 litres of Ethanol per ton (on fermentation and Distillation process).

Excess production of ethanol can be used as Biofuel (anhydrous alcohol, 99.4% -99.9% strength) in blending with petrol (gasoline).

It is therefore desirable to solve current problems, overcome limitations in the prior art and increase Biodiesel production output.

### BRIEF SUMMARY OF THE INVENTION

Accordingly, it is an object of the present invention to provide a method and apparatus for Biodiesel production that utilizes a combination of corn and rice (paddy) as feedstock.

It is another object of the present invention to provide a method and apparatus for Biodiesel production from combination of corn and other natural sources as feedstock.

It is another object of the present invention to provide a method and apparatus for Biodiesel production from combination of corn and Simarouba Seeds and Fruits as feedstock.

It is another object of the present invention to provide a method and apparatus for Biodiesel production from combination of Corn and Mahua seeds (Maduca indica; Maduca longifolia) and dried flowers as feedstock.

It is yet another object of the present invention to provide a method and apparatus for Biodiesel production that uses corn& sweet sorghum.

It is yet another object of present invention to provide a method and apparatus for Biodiesel production from combination of corn and solvent extracted oil from refiners bleach (press) mud as feedstock.

It is still another object of the present invention to provide a method and apparatus for the Biodiesel production that overcomes feedstock shortages, enables stable year round production of Biodiesel, increase equipment utilization rate and delivers economic benefits.

It is still another object of the present invention to provide a method and apparatus for Biodiesel production that reduces the environmentally detrimental effluents from the Biodiesel production processes.

It is yet another object of the present invention to provide a method and apparatus for Biodiesel production that utilizes (unmarketable) damaged paddy, along with corn.

It is yet another object of the present invention to provide a method and apparatus for Biodiesel production that utilizes inedible oil seeds along with corn for Biodiesel production.

It is yet another object of the present invention to provide a method and apparatus for the Biodiesel production that reduces the amount of water required per given amount of ethanol produced in combination of corn and molasses (sugar/molasses).

### DESCRIPTION OF THE ILLUSTRATIVE EMBODIMENT

The primary embodiment of the present invention overcomes the uncertainties in Biodiesel production due to feed stock shortages (scarcity of oil and ethanol) and addresses environmental and water utilization issues by using corn as the basic feed stock and adding Simarouba, Mahua, molasses later in the process.

WET PROCESS: Kernel of corn, as base or major raw material, is inspected and cob, dust, stones, chaff and foreign materials are removed. Cleaning is done before wet processing. Cleaned corn is filled in large steeping tanks (steeps), where corn is soaked in hot water for 40-45 hours.

The steeping is a controlled fermentation and the addition of sulphur dioxide (sulphur stone is heated in oven or SO₂ plant and resultant sulphur dioxide is absorbed in hot water) to the extent of 2000 ppm or lime or both in the hot water (steep water) helps control the fermentation and help suppressing detrimental bacteria, molds, fungi and yeasts. The kernels swells to more than double size, increase moisture content from about 12%-15% to 45% and then kernels softens.

The steep water is drained from the kernels and subjected on multi-stage evaporation. Most of the organic acids formed during steeping process (during fermentation) are volatile and evaporate with water. The hot condensate is used to preheat the (fresh) steep water entering into the steep tanks.

The soften kernels are broken up in attrition/impact mills to loosen the hull and break the bonds between germ and endosperm. Water is added for wet milling process.

The light weight germ ate separated from the ground slurry (by centrifugal force) by/in hydro cyclones (prime germ) and to complete the germ separation process, the slurry is regrounded by second milling followed by second hydrocyclone separation which effectively removes residual germ i.e. secondary germ.

Surface water is removed from the germ and clean germ are dried to approximately 3%-4% moisture in steam dryers (rotary steam tube bundle dryer). Corn oil is extracted from the dried germ using mechanical presses (extruder) and solvent extraction. The crude corn oil is stored for production of Biodiesel. The residual from germ after extraction of oil (germ meal) is stored for animal feeds.

After separation of germ from mill slurry, slurry is finely ground in impact or attrition mills to release starch and gluten from the endosperm (from fibers). The fine slurry is pumped to the screens, where fiber is separated. After fibers screened off, the fine slurry (starch milk) is passed through continuous separators (centrifugal force employed) where gluten and starch is separated.

Gluten slurry is de-watered and dried in steam tube bundle dryers to approximately 8-10% moisture and lumps are disintegrated in a hammer mill.

The dried gluten (60% protein) is mixed with dried fibers and germ meal. The corn gluten meal is packed, for sale as poultry feed.

As the primary objective is to produce ethanol from starch, but not pure starch for industrial purpose (thereby saving water by not washing starch milk again and again), starch milk is pumped to the large storage tanks. From storage tank, starch milk (fine starch slurry) is pumped to Steam Jet Cooker (continuous cooking) where it is cooked upto 105 degree Celsius, the Ph of the starch milk is adjusted to 4.5-5 Ph and cooked starch slurry is passed through holding coil (holding time upto 6-12 minutes) to flash tank. The vapour is routed to condensers in order to condense and the condensate is collected and reused in the process (liquifaction process).

Hot cooked starch milk is cooled down from 105 degree Celsius to 90-95 degree Celsius in heat reduction tank (HRT) which is connected with heat exchanger and pumped to liquefaction reactor where further enzymes are added and if necessary hot process water added in the reactor to dilute the hot cooked starch milk to achieve required viscosity and further heated upto 100 degree Celsius for 30-45 minutes to convert starch milk into simple sugars as much as possible.

This hot starch milk (liquid sugars) is further cooled down to 65-70 degree Celsius in order to further treat it in saccharification vessels where further enzymes added to the treated starch milk, to convert remaining starch into fermentable sugars. On completion of saccharification, saccharified liquid (wash) is pumped to heat exchangers where hot liquid is rapidly cooled down from 65 degree Celsius-70 degree Celsius to 28 degree Celsius-30 degree Celsius.

Wash is transferred to pre-fermentors, nutrients are added to the wash for good, effective fermentation and yeast propagation and yeast multiplication takes place in the pre-fermentors.

After initial fermentation in the pre-fermentors (4-6 hours, sterilized air used) fermented wash is sent to bulk fermentors for completion of fermentation (24-72 hours, continuous or batch process).

Fermentation involves the breaking down of complex organic substances into simpler ones. Alcohol fermentation is enabled by several types of bacteria and yeasts. Through a simple enzymatic anaerobic action, yeasts converts sugar molecules into alcohol (ethanol) and carbon dioxide. The above reaction is simplified one that actually. involves intermnediate steps—that of the generation of pyruvic acid, its conversion into acetaldehyde and finally to ethanol.

Fermented wash is subjected to a distillation process (fractional distillation) in distillation columns and ethanol is produced (94.68% to 95%).

Ethanol further dehydrated in molecular sieve where the strength of ethanol reaches 99.8% to 99.9% after water content is reduced. This fuel ethanol is stored and used for production of Biodiesel.

Crude corn oil (derived from corn germ), ethanol plus base catalyst sodium hydroxide or potassium hydroxide are fed into transesterification plant (batch or continuous) to produce crude Biodiesel and by-product crude Glycerin. Crude Biodiesel is further refmed and clean biodiesel produced. Crude Glycerin can be further refined for produce Glycerin (99% pure) or can be disposed off as Crude Glycerin. (This process is further explained under dry process of corn)

DRY PROCESS: Instead of Wet Milling Process of Corn, bran & germ can be separated in dry milling process where very less water is consumed and treatment of effluent water is also reduced compare to wet process. Kernel of corn, base or major raw material, is inspected and cob, dust, stones, chaff and foreign materials are removed. Cleaning is done before dry processing.

In dry process, moisture is applied uniformly to each corn (maize) kernel, thereby penetration of moisture through the bran skin and into the germ takes place. By controlling the quantity of moisture added to the grain and retention time of the grain, efficient pre-conditioning can be achieved in less time than the other conventional methods. Pre-conditioning helps to separate bran and germ from the core endosperm of the corn (maize) kernel.

Pre-conditioned kernel is fed to the de-germinator machine/plant, where under aggressive rubbing action (grain against grain), both bran and germ from individual corn (maize) kernels removed simultaneously.

Germ & bran separated from large pieces of pure endosperm through separate screens. Germ separated from kernel, if necessary, dried in steam dryers (rotary steam tube bundle dryer) to maintain moisture level of 3-4%.

Corn oil is extracted from the dried germs using mechanical process (extruders) and solvent extraction. The crude corn oil is stored for production of Biodiesel.

The residual from germ after extraction of oil (germ meal) is mixed with corn bran (separated from kernel) and is stored for animal/poultry feeds.

Pieces of endosperm, free from bran and germ is fed to hammer mill/grinding mill, where it is further reduced to required size (corn flour). From hammer mill/grinding mill, the corn flour is conveyed to turbo sieves, where it is screened (fine flour, below 0.2 mm is separated) and 0.2 mm to 0.6 mm corn flour is mixed with hot water in slurry tanks and agitated in reactors where steam is applied and Enzymes added to convert the starch into dextrin (a type of glucose) before pumped to steam jet cooker (continuous cooking), where it is cooked upto 105 degree Celsius, the Ph of the slurry is adjusted to 4.5-5 Ph and the cooked starch slurry is pumped through holding coil (holding time 6-12 minutes) to flash tank.—(In recent developments special enzymes were added in reactor vessels to starch slurry and treated with low temperature (say 70 degree Celsius-90 degree Celsius) for 60-90 minutes to convert starch into low dextrin whereby jet cooking is avoided).

The vapour is routed to condensers in order to condense and the condensate is collected and reused in the process (liquifaction process).

Hot cooked slurry i.e. cooked starch milk is cooled down to 105 degree Celsius to 90-95 degree Celsius in heat reduction tank (HRT) which is connected with heat exchanger and pumped to liquifaction reactor where further enzymes are added and if necessary hot process water added in the reactor to dilute the hot cooked starch milk slurry to achieve required viscosity and further heated for 30-45 minutes (upto 100 degree Celsius) to convert starch milk into simple sugars.

This hot starch milk slurry (liquid sugar) is further rapidly cooled down to 65-70 degree Celsius in order to further treat it in saccharification vessels where further enzymes added to starch milk, to convert remaining starch into fermentable sugars.

On completion of saccharification, saccharified liquid (wash) is pumped to heat exchangers where hot liquid is cooled down rapidly from 65 degree Celsius-70 degree Celsius to 28 degree Celsius-30 degree Celsius.

Wash is transferred to pre-fermentors, where distilleries yeast and nutrients are added to the wash for good, effective yeast propagation and fermentation. Yeast multiplication takes place in the pre-fermentors.

After initial fermentation and yeast multiplication in the pre-fermentors (4-6 hours, sterilized air used) fermented wash is sent to bulk fermentors for completion of fermentation (24-72 hours).

Fermentation involves the breaking down of complex organic substances into simpler ones. Alcohol fermentation is enabled by several types of bacteria and yeasts. Through a simple enzymatic anaerobic action, yeasts converts sugar molecules into alcohol (ethanol) and carbon dioxide. The above reaction is simplified one that actually involves intermediate steps—that of the generation of pyruvic acid, its conversion into acetaldehyde and finally to ethanol.

Fermented wash is subjected to fractional distillation process in distillation columns and ethanol is produced (94.68% to 95%).

Ethanol further dehydrated in molecular sieve to reduce the water content in the ethanol and the strength of ethanol reaches 99.8% to 99.9%, which is used for production of Biodiesel.

Biodiesel is produced with corn oil/corn oil with other oil mixture, with ethanol and base catalyst in transesterification process at low temperature and pressure.

The catalyst is typically sodium hydroxide (caustic soda) or potassium hydroxide (potash). Catalyst and ethanol is fed into reactor (agitator) or mixer where catalyst is dissolved in the Ethanol (alcohol).

Corn oil or corn oil mixed with other oils is first heated to 100-102 degree Celsius to remove water component and cooled to the desired level. The cooled oil is filtered to remove contaminants. Water is removed to prevent soap formation. Free fatty acids level also kept below 15% to prevent soap formation and avoid problems in separation of glycerin (by-product).

The ethanol catalyst mix is fed into closed reaction vessel, also known as transesterification plant and corn oil or corn oil mixed with other oils (either edible or inedible) charged into the closed vessel (closed to the atmosphere in order to prevent loss of ethanol).

Ethanol (99.8% to 99.9%) reduces viscosity of oil & fats. Catalyst sodium hydroxide (caustic soda) or potassium hydroxide is used to convert TG's (triglycerides) into Alkyl Esters. Transesterification of corn oil (maize oil) or corn oil mixed with other oils (either edible or inedible) with ethanol on the molar ratio of ethanol to corn oil at 30:1 as first order and 6:1 as second order, which is reverse reaction. The mix is heated upto just above the ethanol boiling point (68-74 degree Celsius) to speed up the reaction and the reaction takes place. At the completion of the reaction (transesterification) which may varies from 2-8 hours at room temperature, the oil molecules (TG's) are broken apart to reform into ethylesters (Biodiesel) and glycerin. Both products i.e. crude biodiesel and crude glycerin is separated by gravity since crude glycerin is more dense than crude biodiesel. Crude glycerin is separated from the bottom of the transesterification vessel.

For faster separation the entire mix is drained into neutralization tanks where citric acid solution is added for neutralization and centrifuge is used to separate crude biodiesel and glycerin.

The separated crude biodiesel & glycerin, have excess ethanol, which is removed by distillation process and reused.

The glycerin by-product contains soaps and unused catalyst sodium hydroxide or potassium hydroxide which again re-neutralized using acid (citric acid) to remove the residual contaminate. This glycerin is subjected to distillation to remove excess ethanol and the resulted crude glycerin (80-88% pure) is stored.

If necessary the crude glycerin 80-88% can be further purified to the purity level of 99%.

Separated crude biodiesel has excess ethanol which is removed by distillation process and reused.

Separated biodiesel from glycerin is purified to remove residual soaps or catalyst, by using warm water (by gentle washing) and dried and transferred to storage tanks by using pumps.

In another embodiment of the present invention, Simarouba [simarouba glauca DC] oil seed which has about 60%-70% in higher oil content (edible) is separated from Simarouba ripened fruits and added to corn germ obtained from corn processing (wet or dry milling process) for extracting crude oil to use in the production of biodiesel. Simarouba fruits, which contains 11% sugars is crushed to produce pulp. Process water added to pulp, with enzymes and cooked in pasteurization plant upto 105 degree Celsius where fructose and other sugars were converted into fermentable sugars. The Ph of the juice is adjusted to 4.5-5 Ph. This hot syrup is transferred to flash tank where vapour is routed to condensers in order to condense and the condensate is collected and reused as process water. From flash tank the syrup is pumped to heat reduction tanks (HRT) which is connected with heat exchangers where the syrup is rapidly cooled down from 105 degree Celsius to 28-30 degree Celsius. This cooled syrup is added to saccharified wash (corn wash) using static mixture machine (upto 50% v/v). This wash (combination of corn wash & Simarouba fruit syrup) is treated in pre-fermentors and fermented to produce ethanol (at lower cost).

In another embodiment of the present invention, Mahua seeds (Maduca indica; Maduca longifolia), which has about 34% to 38% in higher oil content is added to germ obtained from corn processing for extracting crude oil by using mechanical process (extruders) and solvent process, to use in the production of Biodiesel.

In another embodiment of the present invention, Mahua seeds (Maduca indica; Maduca longifolia), which has about 34% to 38% in higher oil content is added to germ obtained from corn processing for extracting crude oil by using mechanical process (extruders) and Mahua syrup is added to the corn starch milk (after saccharification) for production of Biodiesel.

Mahua syrup is produced from dried mahua flowers (madhuca latifolia and madhuca longifolia (Koenig) Macbrim; synonyms bassia longifolia, illipe longifolia) which contains sugars up to 60-70%. Dried flowers are crushed and hot process water is added in the pasteurization vessel and agitated. Steam is used for heating, the enzymes are added to convert fructose, sucrose and other sugars into fermentable sugars and the mixture is boiled upto 105 degree Celsius. The Ph of the liquid (syrup) is adjusted to 4.5-5 Ph. The syrup with brix reading of 45 is transferred to heat reduction tank, which is connected with heat exchangers where the temperature is rapidly reduced from 105 degree Celsius to 28-30 degree Celsius. This cooled syrup is transferred to a static mixture where saccharrified starch milk is mixed to increase the sugar content of the wash upto 33%. This wash is further subjected to fermentation and distillation process to produce ethanol which is used for biodiesel production.

In another embodiment of the present invention, Jatropha seeds (jatropa curcas; wild caster seeds), which has about 30% to 33% oil added to germ obtained from corn processing for extracting crude oil by using mechanical process (extruders) and solvent process, to use in the production of Biodiesel.

In another embodiment of the present invention, Pongamia seeds (pongamia pinnata (L) pierre, pongamia glabra vent, pongam karanga) which has about 30% to 33% oil content, added to germ obtained from corn processing for extracting crude oil by using mechanical process (extruders) and solvent process, to use (as feedstock) in the production of Biodiesel.

In another embodiment of present invention, peanut oil seeds (kernels) which has higher oil content (24%-25%) added to germ obtained from corn processing for extraction of crude oil by using mechanical process (extruders) and solvent process, to use in the production of Biodiesel.

In another embodiment of present invention, Canola oil seeds which has higher oil content (38%-40%) are added to germ obtained from corn processing for extraction of crude oil by using mechanical process (extruders) and solvent process, to use in the production of Biodiesel.

In another embodiment of present invention, Rapeseed (oil seed) which has 38%-40% oil content, is added to germ obtained from corn processing for extraction of crude oil by using mechanical process (extruders) and solvent process, to use in the production of Biodiesel.

In another embodiment of present invention, Cotton seeds which has 17%-19% oil content, is added to germ obtained from corn processing for extraction of crude oil by using mechanical process (extruders) and solvent process, to use in the production of Biodiesel.

In another embodiment of present invention, Sunflower seeds which has 35%-40% oil content, is added to germ obtained from corn processing for extraction of crude oil by using mechanical process (extruders) and solvent process, to use in the production of Biodiesel.

In another embodiment of present invention, Mustard Oil seeds (spice, hybrids) which has 35%-40% oil content, is added to germ obtained from corn processing for extraction of crude oil by using mechanical process (extruders) and solvent process, to use in the production of Biodiesel.

In another embodiment of present invention, Safflower seeds which has 23%-25% oil content, is added to germ obtained from corn processing for extraction of crude oil by using mechanical process (extruders) and solvent process, to use in the production of Biodiesel.

In another embodiment of present invention, Castor Oil seeds which has 20%-40% oil content, is added to germ obtained from corn processing for extraction of crude oil by using mechanical process (extruders) and solvent process, to use in the production of Biodiesel.

In another embodiment of present invention, Neem seeds (margosa) which has 15%-25% oil content, is added to germ obtained from corn processing for extraction of crude oil by using mechanical process (extruders) and solvent process, to use in the production of Biodiesel.

In another embodiment of present invention, Crude Palm Oil is added to crude corn oil obtained from germ (from corn processing) and sweet sorghum (sorghum bicolor (L) moench) flour is added to starch milk (slurry) obtained from corn processing.

In another embodiment of present invention, Crude Palm Oil is added to crude corn oil obtained from germ (from corn processing wet/dry) and Simarouba Fruit Syrup is added to starch milk (after saccharification) obtained from corn processing.

In another embodiment of present invention, Crude Soy Oil is added to crude corn oil obtained from germ (from corn processing) and sweet sorghum (sorghum bicolor (L) moench) flour is added to starch milk (starch slurry) obtained from corn processing.

In another embodiment of present invention, Crude Soy Oil is added to crude corn oil obtained from germ (from corn processing wet/dry) and Simarouba Fruit Syrup is added to starch milk (after saccharification) obtained from corn processing.

In another embodiment of present invention, Crude Mahua Oil (Maduca indica; Maduca longifolia) is added to crude corn oil obtained from germ (from corn processing) and sweet sorghum (sorghum bicolor (L) moench) flour is added to starch milk (starch slurry) obtained from corn processing.

In another embodiment of present invention, Crude Mahua Oil (Maduca indica; Maduca longifolia) is added to crude corn oil obtained from germ (from corn processing wet/dry) and Simarouba Fruit Syrup is added to starch milk (after saccharification) obtained from corn processing.

In another embodiment of present invention, Crude Mahua Oil (Maduca indica; Maduca longifolia) is added to crude corn oil obtained from germ (from corn processing wet/dry) and Simarouba Fruit Syrup and Mahua Syrup is added to starch milk (after saccharification) obtained from corn processing.

In another embodiment of present invention, Jatropha Oil (jatropa curcas; wild caster seeds) (inedible) is added to corn oil obtained from germ (from corn processing wet/dry) and Simarouba Fruit Syrup is added to starch milk (after saccharification) obtained from corn processing.

In another embodiment of present invention, Jatropha Oil (jatropa curcas; wild caster seeds) (inedible) is added to corn oil obtained from germ (from corn processing) and sweet sorghum (sorghum bicolor (L) moench) flour is added to starch milk (starch slurry) obtained from corn processing.

In another embodiment of present invention, Pongamia Oil (pongamia pinnata (L) pierre, pongamia glabra vent, pongam karanga) (inedible) is added to crude corn oil obtained from germ (from corn processing) and sweet sorghum (sorghum bicolor (L) moench) flour is added to starch milk (starch slurry) obtained from corn processing.

In another embodiment of present invention, Pongamia Oil (pongamia pinnata (L) pierre, pongamia glabra vent, pongam karanga) (inedible) is added to crude corn oil obtained from germ (from corn processing wet/dry) and Simarouba Fruit Syrup is added to starch milk (after saccharification) obtained from corn processing.

In another embodiment of present invention, Simarouba Oil is added to crude corn oil obtained from germ (from corn processing) and Simarouba fruit syrup is added to the starch milk (after saccharification) obtained from corn processing (WET/DRY PROCESSING).

In another embodiment of present invention, Simarouba Oil is added to crude corn oil obtained from germ (from corn processing) and Simarouba fruit syrup and Mahua Syrup is added to the starch milk (after saccharification) obtained from corn processing (wet/dry processing).

In another embodiment of present invention, Straight Vegetable Oil (SVO) is added to corn oil obtained from germ (from corn processing) for production of Biodiesel.

In another embodiment of present invention, Neem Oil (margosa) is added to crude corn oil obtained from germ (from corn processing) for production of Biodiesel.

In another embodiment of present invention, Mahua Oil (Maduca indica; Maduca longifolia) is added to crude corn oil obtained from germ (from corn processing) for production of Biodiesel.

In another embodiment of present invention, Mahua Oil (Maduca indica; Maduca longifolia) is added to crude corn oil and Mahua Syrup added to starch milk (after saccharification) obtained from germ (from corn processing) for production of BioDiesel.

In another embodiment of present invention, Rice Bran Oil added to crude corn oil obtained from germ (from corn processing) for production of Biodiesel.

In another embodiment of present invention, waste oil obtained from refiners bleach (pressmud) mud added to crude corn oil obtained from germ (from corn processing). In edible oil refinery industry huge quantities of spent bleach mud accumulating everyday and disposal of this spent mud which contains 18-20% fats and oil has become a huge problem and pollution created by this spent mud during rainy season etc. is attracting heavy penalty from pollution control departments. These refinery industries finding it difficult to dispose this pollutant bleach mud. To mitigate this problem and extract the oil from the spent bleach mud and use it in useful manner, this process has been invented to convert this oil into Biodiesel.

In the edible oil industry refining edible oil, sodium bentonite (bentonite clay powder) is used for neutralizing and removing fats. After using the bentonite for process the bentonite is filtered and thrown away as a waste product. During the process of refining the bentonite absorbs the fats and oil to the extent of 18-20%. The waste spent bleach mud which contains 18-20% of oil & fats become hard lumps.

This hard lumps are fed into hammer mill to break into small pieces and transferred to process vessel. Industrial Solvent (Hexane, normal) is added to the spent bleach mud and agitated. The ratio (atleast) solvent to spent bleach mud is 10:1 in the first reactor vessel and allowed to dissolved oils and fats in the solvents. Agitation takes place for 15-20 minutes until it become fine slurry. This fine slurry is transferred through screw conveyor which passes through series of filters. Solvent rich with oil is separated from the spent bleach mud. The separated solvent oil mixture is collected in a tank and passed to another reactor vessel. Here further solvent is added to dilute the liquid and agitated for 10-15 minutes. Then this liquid further passed through membranes filters to remove spent bleach mud and the liquid is collected in another tank and pumped to a jacketed vessel, where steam is passed through the jacket and the liquid (oil with solvent) is heated indirectly upto 60-70 degree Celsius to evaporate the solvent.

The solvent is evaporated, the vapour fed to condenser where the vapour is condensed and condensate solvent collected for the reuse. Oil is separated from the solvent and rapidly cooled from 60-70 degree Celsius to room temperature (30-32 degree Celsius) the cooled oil again subjected to filtration for final use. This inedible oil is mixed with corn oil for production of Biodiesel.

In another embodiment of present invention, Rice Bran Oil added to crude corn oil and Rice Flour (from damaged rice) is added to starch milk (starch slurry) obtained from from corn processing for economical production of Biodiesel.

In another embodiment of present invention, Rice Bran Oil added to crude corn oil and Molasses added to starch milk (starch slurry) obtained from corn processing for economical production of Biodiesel.

In another embodiment of present invention, Straight Vegetable Oil added to crude corn oil and molasses added to starch milk (starch slurry) obtained from corn processing for economical production of Biodiesel.

In another embodiment of present invention, Jatropha oil (jatropa curcas; wild caster seeds) (inedible) added to crude corn oil and molasses added to starch milk (starch slurry) obtained from corn processing for economical production of Biodiesel.

In another embodiment of present invention, Mahua Oil (Maduca indica; Maduca longifolia) (inedible) is added to crude corn oil obtained from germ (from corn processing) and molasses added to starch milk (starch slurry) obtained from corn processing for economical production of Biodiesel.

In another embodiment of present invention, Pongamia Oil (pongamia pinnata (L) pierre, pongamia glabra vent, pongam karanga) (inedible) is added to crude corn oil obtained from germ and molasses added to starch milk (starch slurry) obtained from corn processing for economical production of Biodiesel.

In the foregoing conclusion, the invention has been described with reference to illustrative embodiments thereof. However, it will be evident that various modifications and changes may be made thereto without departing from the broader spirit and scope of the invention. The specifications and drawings are, accordingly, to be regarded in an illustrative rather than a restrictive sense. Therefore, it is object of the appended claims to cover all such modifications and changes as come within the true spirit and scope of invention.

1. A system of economical production of biodiesel from combination of corn and other agro feedstock, wherein:
the base or major raw material is corn (maize) and agro feedstock is simarouba,
wherein corn germ and simarouba seeds are mixed and oil extracted, followed by corn starch converted into fermentable sugars by liquification & saccharification process. Simarouba fruit syrup mixed with corn starch after saccharification process,
wherein yeast is added to the mixture and fermentation takes place for about 72 hours, where after fermented wash is distilled to produce ethanol,
wherein corn oil & simarouba oil mixture, ethanol and catalyst are subjected to transesterification process to produce biodiesel and by-product glycerin.

2. A system of claim 1, where agro feedstock is mahua seeds and mahua dried flowers.

3. A system of claim 1, where agro feedstock is simarouba and mahua seeds and syrup.

4. A system of claim 1, where agro feedstock is pongamia seeds.

5. A system of claim 1, where agro feedstock is neem seeds.

6. A system of economical production of biodiesel from combination of corn, and unedible oil extracted from spent bleach mud (spent sodium bentonite mud), wherein the base or major raw material is corn.

7. A method of economical production of biodiesel from combination of corn and other agro feedstock, wherein:
the base or major raw material is corn (maize) and agro feedstock is simarouba,
wherein corn germ and simarouba seeds are mixed and oil extracted, followed by corn starch converted into fermentable sugars by liquification & saccharification process. Simarouba fruit syrup mixed with corn starch after saccharification process,
wherein yeast is added to the mixture and fermentation takes place for about 72 hours, where after fermented wash is distilled to produce ethanol,
wherein corn oil & simarouba oil mixture, ethanol and catalyst are subjected to transesterification process to produce biodiesel and by-product glycerin.

8. A method of claim 7, where agro feedstock is mahua seeds and mahua dried flowers.

9. A method of claim 7, where agro feedstock is simarouba and mahua seeds and syrup.

10. A method of claim 7, where agro feedstock is pongamia seeds.

11. A method of claim 7, where agro feedstock is neem seeds.

12. A method of economical production of biodiesel from combination of corn, and unedible oil extracted from spent bleach mud (spent sodium bentonite mud), wherein the base or major raw material is corn.
